(19) United States
(12) Patent Application Publication
(10) Pub. No.: US 2023/0039711 A1
(43) Pub. Date: Feb. 9, 2023

(54) AHR INHIBITORS AND USES THEREOF

(71) Applicant: Ikena Oncology, Inc., Boston, MA (US)

(72) Inventors: Marta SANCHEZ-MARTIN, Somerville, MA (US); Lei WANG, Braintree, MA (US); Xiaoyan Michelle ZHANG, Lexington, MA (US)

(21) Appl. No.: 17/791,608

(22) PCT Filed: Jan. 8, 2021
(86) PCT No.: PCT/US2021/012571

§ 371 Date: Jul. 8, 2022

Related U.S. Application Data
(60) Provisional application No. 63/128,465, filed on Dec. 21, 2020.
(60) Provisional application No. 62/959,246, filed on Jan. 10, 2020.

(51) Int. Cl. A61K 31/53; A61P 35/00

(52) U.S. Cl. CPC A61K 31/53; A61P 35/00

(57) ABSTRACT

The present invention provides methods for selecting a cancer patient who is AHR nuclear positive, and methods for treating cancer comprising selecting a cancer patient who is AHR nuclear positive, and administering to the patient an AHR inhibitor.

### CROSS-REFERENCE TO RELATED APPLICATIONS

This application claims the benefit under 35 U.S.C. § 119(e) of U.S. Provisional Pat. Application No. 62/959,246, filed Jan. 10, 2020; and U.S. Provisional Pat. Application No. 63/128,465, filed Dec. 21, 2020, the contents of each of which are herein incorporated by reference in their entireties.

### TECHNICAL FIELD OF THE INVENTION

The present invention relates to uses of AHR inhibitors for treating cancer patients who are AHR nuclear positive.

### BACKGROUND OF THE INVENTION

Aryl hydrocarbon receptor (AHR) is a ligand-activated nuclear transcription factor that, upon binding to ligand, translocates from the cytoplasm to the nucleus and forms a heterodimer with aryl hydrocarbon receptor nuclear translocator (ARNT) (Stevens, 2009). The AHR-ARNT complex binds to genes containing dioxin response elements (DRE) to activate transcription. Numerous genes are regulated by AHR; the most well documented genes include the cytochrome P450 (CYP) genes, CYP1B1 and CYP1A1 (Murray, 2014).

Multiple endogenous and exogenous ligands are capable of binding to and activating AHR (Shinde and McGaha, 2018; Rothhammer, 2019). One endogenous ligand for AHR is kynurenine, which is generated by indoleamine 2, 3-dioxygenase 1 (IDO1) and tryptophan 2,3-dioxygenase (TDO2) from the precursor tryptophan. Many cancers over-express IDO1 and/or TDO2, leading to high levels of kynurenine. Activation of AHR by kynurenine or other ligands alters gene expression of multiple immune modulating genes leading to immunosuppression within both the innate and adaptive immune system (Opitz, 2011). Activation of AHR leads to differentiation of naive T cells toward regulatory T cells (Tregs) over effector T cells (Funatake, 2005; Quintana 2008). It has recently been shown that activated AHR up-regulates programmed cell death protein 1 (PD-1) on CD8+ T cells to reduce their cytotoxic activity (Liu, 2018). In myeloid cells, AHR activation leads to a tolerogenic phenotype on dendritic cells (Vogel, 2013). In addition, AHR activation drives the expression of KLF4 that suppresses NF-κB in tumor macrophages and promotes CD39 expression that blocks CD8+ T cell function (Takenaka, 2019).

AHR-mediated immune suppression plays a role in cancer since its activity prevents immune cell recognition of and attack on growing tumors (Murray, 2014; Xue, 2018; Takenaka, 2019).

### SUMMARY OF THE INVENTION

As described herein, the inventors have discovered that AHR nuclear localization and/or AHR gene amplification are indicative of patient responsiveness to treatment with an AHR inhibitor or AHR antagonist. Surprisingly, it was found that the percentage of AHR nuclear positive patients varies significantly across different types of cancer. For example, it was determined that there are a higher percentage of bladder cancer patients who are AHR nuclear positive than other cancer types. Some AHR inhibitors, such as (R)-N-(2-(5-fluoropyridin-3-yl)-8-isopropylpyrazolo[1,5-a][1,3,5]triazin-4-yl)-2,3,4,9-tetrahydro-1H-carbazol-3-amine (Compound A), can block AHR from translocating from the cytoplasm to the nucleus in the presence of a ligand and can block downstream signaling in in vivo tumor models. Accordingly, for certain cancer types (such as, for example, bladder cancer), determining AHR nuclear positivity and/or AHR gene amplification can be used to determine or predict efficacy of treatments using AHR antagonists, and for patient selection purposes.

Accordingly, provided herein, are methods for determining or predicting efficacy of treatments using AHR antagonists and/or selecting a patient for application or administration of a treatment comprising an AHR antagonist, such as Compound A. Such methods comprise, in part, methods of identifying patients having AHR nuclear positivity and/or AHR gene amplification, and methods for treating patients having AHR nuclear positivity and/or AHR gene amplification using AHR antagonists, such as Compound A.

Provided herein are methods for identifying cancer patients who are AHR nuclear positive, and uses of an AHR inhibitor for treating cancer patients who are AHR nuclear positive.

In one aspect, the present invention provides a method for identifying or selecting a cancer patient who is AHR nuclear positive, comprising immunohistochemistry (IHC) staining a tumor tissue of a patient, and selecting a patient who is AHR nuclear staining positive.

In another aspect, the present invention provides a method of treating cancer, comprising selecting a patient who is AHR nuclear positive, and administering to the patient a therapeutically effective amount of an AHR inhibitor.

In some aspects and embodiments, the present invention provides a method of treating a proliferative disorder, such as cancer, in a patient, comprising selecting a patient who is AHR nuclear positive, for example, using a method as described herein, and administering to the patient a therapeutically effective amount of an AHR antagonist, for example, as described herein. In some embodiments, a treatment method further comprises measuring or determining whether a tumor sample from the patient is AHR nuclear positive, for example, using an IHC staining method as described herein.

In some aspects and embodiments, the present invention provides a method of treating a proliferative disorder, such as cancer, in a patient, comprising selecting a patient who has an AHR gene amplification, for example, using a method as described herein, and administering to the patient a therapeutically effective amount of an AHR antagonist, for example, as described herein. In some embodiments, a treatment method further comprises measuring or determining whether a tumor sample from the patient has an AHR gene amplification, for example, using any of the methods as described herein, for example, NGS, RNAscope, or FISH.

In some aspects and embodiments, the present invention provides a method of treating a proliferative disorder, such as cancer, in a patient who is AHR nuclear positive, comprising administering to the patient a therapeutically effective amount of an AHR antagonist, for example, as described herein. In some embodiments, the patient is determined as being AHR nuclear positive, for example, using a method as described herein. In some embodiments, a treatment method further comprises measuring or determining whether a tumor sample from the patient is AHR nuclear positive, for example, using an IHC staining method as described herein.

In some aspects and embodiments, the present invention provides a method of treating a proliferative disorder, such as cancer, in a patient who has an AHR gene amplification, comprising administering to the patient a therapeutically effective amount of an AHR antagonist, for example, as described herein. In some embodiments, the patient is determined as having an AHR gene amplification, for example, using a method as described herein. In some embodiments, a treatment method further comprises measuring or determining whether a tumor sample from the patient has an AHR gene amplification, for example, using any of the methods as described herein, for example, NGS, RNAscope, or FISH.

In some embodiments, a cancer is selected from those as described herein. In some embodiments, an AHR inhibitor is selected from those as described herein. In some embodiments of these methods, the AHR antagonist is Compound A, or a pharmaceutically acceptable salt thereof. In some embodiments of these methods, the AHR antagonist is a metabolite of Compound A, or a pharmaceutically acceptable salt thereof, or a prodrug thereof. In some embodiments, a metabolite of Compound A is Compound B or Compound C.

### BRIEF DESCRIPTION OF FIGURES

FIG. 1 depicts bladder cancer CTA scoring of AHR nuclear staining at all intensity (A) and at pooled 2+ 3+ intensity (B).

FIG. 2 depicts melanoma TMA (811) CTA scoring of AHR nuclear staining at all intensity (A) and at pooled 2+ 3+ intensity (B).

FIG. 3 depicts melanoma TMA (804b) CTA scoring of AHR nuclear staining at all intensity (A) and at pooled 2+ 3+ intensity (B).

FIG. 4 depicts ovarian cancer CTA scoring of AHR nuclear staining at all intensity (A) and at pooled 2+ 3+ intensity (B).

FIG. 5 depicts HNSCC CTA scoring of AHR nuclear staining at all intensity (A) and at pooled 2+ 3+ intensity (B).

FIG. 6 depicts H-scores of bladder cancer, melanoma, ovarian cancer, and HNSCC. The lines represent the mean values.

### DETAILED DESCRIPTION OF THE INVENTION

### 1. General Description of Certain Embodiments of the Invention

As described herein, it has been found that AHR nuclear localization and/or AHR gene amplification can be used as a predictive biomarker for identifying and selecting cancer patients who can receive clinical benefit or be responsive to treatment with an AHR inhibitor, such as (R)-N-(2-(5-fluoropyridin-3-yl)-8-isopropylpyrazolo[1,5-a][1,3,5]triazin-4-yl)-2,3,4,9-tetrahydro-1H-carbazol-3-amine (Compound A).

It has been found that AHR inhibitor (R)-N-(2-(5-fluoropyridin-3-yl)-8-isopropylpyrazolo[1,5-a][1,3,5]triazin-4-yl)-2,3,4,9-tetrahydro-1H-carbazol-3-amine (Compound A) effectively blocks AHR from translocating from the cytoplasm to the nucleus in the presence of a ligand and downstream signaling in in vivo tumor models. Compound A is a novel, synthetic, small molecule inhibitor designed to target and selectively inhibit the AHR and is being developed as an orally administered therapeutic. It has been found that there are multiple tumor types that have high levels of AHR signaling as determined by an AHR-gene signature. The high level of AHR activation caused by elevated levels of kynurenine and other ligands, as well as its role in driving an immune suppressive tumor microenvironment (TME), make AHR an attractive therapeutic target in multiple cancer types.

Compound A potently inhibits AHR activity in human and rodent cell lines (~35-150 nM half maximal inhibitory concentration [IC50]) and is highly selective for AHR over other receptors, transporters, and kinases. In human T cell assays, Compound A induces an activated T cell state. Compound A inhibits CYP1A1 and interleukin (IL)-22 gene expression and leads to an increase in pro-inflammatory cytokines, such as IL-2 and IL-9.

The nonclinical safety of Compound A has been evaluated in a series of pharmacological, single-dose and repeated-dose toxicological studies in rodent and non-rodent species including 28-day Good Laboratory Practice (GLP) studies in rat and monkeys. Noteworthy findings in these studies of potential relevance to humans included: emesis, loose stool, dehydration, body weight loss, nonglandular stomach ulceration and edema (rats), seminiferous tubule degeneration and debris in the epididymis lumen (rats), up to 11% QTc prolongation (monkeys) and decreased thymus weights and cortical lymphocytes (monkey). All changes were resolved or resolving after 2 weeks of dosing cessation, except for the testicular changes in rats. The nonclinical safety assessment from these studies supports clinical evaluation of Compound A in humans. Doses of 200 mg, 400 mg, 800 mg, and 1200 mg once daily (QD) of Compound A have been tested in human patients with no serious adverse events (SAEs) as a monotherapy.

It has also been found that immunohistochemistry (IHC) staining can identify cancer patients who are AHR nuclear positive. Various tumor tissues have been analyzed using immunohistochemistry (IHC) staining. See, for example, the IHC staining data of bladder cancer, melanoma, ovarian cancer, and head and neck squamous cell carcinoma (HNSCC), as described herein. Without wishing to be bound by any particular theory, cancer patients who are AHR nuclear positive are more likely to benefit from an AHR inhibitor treatment.

Surprisingly, it was found that the percentage of AHR nuclear positive patients varies significantly across different types of cancer. For example, based on IHC staining, there are a higher percentage of bladder cancer patients who are AHR nuclear positive than other cancer types. Accordingly, for certain cancer types (for example, bladder cancer), a preselection of AHR nuclear positive patients can significantly enhance the effectiveness of an AHR inhibitor treatment.

Accordingly, in some aspects, the present invention provides a method of treating a proliferative disorder, such as cancer, in a patient, comprising selecting a patient who is AHR nuclear positive, for example, using a method as described herein, and administering to the patient a therapeutically effective amount of an AHR antagonist, for example, as described herein. In some embodiments, a treatment method further comprises measuring or determining whether a tumor sample from the patient is AHR nuclear positive, for example, using an IHC staining method as described herein.

In some aspects, the present invention provides a method of treating a proliferative disorder, such as cancer, in a patient, comprising selecting a patient who has an AHR gene amplification, for example, using a method as described herein, and administering to the patient a therapeutically effective amount of an AHR antagonist, for example, as described herein. In some embodiments, a treatment method further comprises measuring or determining whether a tumor sample from the patient has an AHR gene amplification, for example, using any of the methods as described herein, for example, NGS, RNAscope, or FISH.

In some aspects, the present invention provides a method of treating a proliferative disorder, such as cancer, in a patient who is AHR nuclear positive, comprising administering to the patient a therapeutically effective amount of an AHR antagonist, for example, as described herein. In some embodiments, the patient is determined as being AHR nuclear positive, for example, using a method as described herein. In some embodiments, a treatment method further comprises measuring or determining whether a tumor sample from the patient is AHR nuclear positive, for example, using an IHC staining method as described herein.

In some aspects, the present invention provides a method of treating a proliferative disorder, such as cancer, in a patient who has an AHR gene amplification, comprising administering to the patient a therapeutically effective amount of an AHR antagonist, for example, as described herein. In some embodiments, the patient is determined as having an AHR gene amplification, for example, using a method as described herein. In some embodiments, a treatment method further comprises measuring or determining whether a tumor sample from the patient has an AHR gene amplification, for example, using any of the methods as described herein, for example, NGS, RNAscope, or FISH.

Accordingly, in one aspect, the present invention provides a method for IHC staining a tumor tissue of a patient, comprising staining a tumor tissue section using an AHR monoclonal antibody. In another aspect, the present invention provides a method for identifying or selecting a cancer patient, comprising using an IHC staining as described herein. In another aspect, the present invention provides a method for treating cancer, comprising selecting a cancer patient using an IHC staining as described herein, and administering a therapeutically effective amount of an AHR inhibitor as described herein.

### 2. Definitions

As used herein, the term “an AHR inhibitor” refers to a compound, or a pharmaceutically acceptable salt or ester thereof, which inhibits AHR activity in a biological sample or in a patient. An AHR inhibitor, also referred to herein as an AHR antagonist, can bind but does not activate the AHR polypeptide or polynucleotide encoding the AHR, and the binding disrupts the interaction, displaces an AHR agonist, and/or inhibits the function of an AHR agonist. An AHR inhibitor, or an AHR antagonist, can include small molecules (organic or inorganic), proteins, such as antagonistic anti-AHR antibodies, nucleic acids, amino acids, peptides, carbohydrates, or any other compound or composition which decreases the activity of AHR, either by reducing the amount of AHR present in a cell, or by decreasing the binding or signaling activity or biological activity of AHR, such as by, for example, blocking AHR from translocating from the cytoplasm to the nucleus in the presence of a ligand and/or blocking downstream signaling activities. Various AHR antagonists have been described previously, for example, in WO2017202816A1, WO2018085348A1, WO2018195397, WO2019101642A1, WO2019101643A1, WO2019101641A1, WO2019101647A1, WO2019036657A1, US10570138B2, US10689388B1, US10696650B2, WO2020051207A2, WO2020081636A1, and WO2020081840A1, the contents of each of which are incorporated herein by reference in their entireties, and others are described herein.

As used herein, the term “Compound A” refers to an AHR inhibitor, (R)-N-(2-(5-fluoropyridin-3-yl)-8-isopropylpyrazolo[1,5-a][1,3,5]triazin-4-yl)-2,3,4,9-tetrahydro-1H-carbazol-3-amine, of formula:

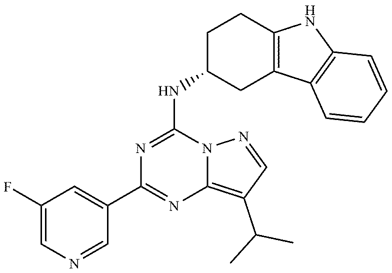 [SMILES: CC(C)c1cnn2c(N[C@@H]3CCc4[nH]c5ccccc5c4C3)nc(-c3cncc(F)c3)nc12]

In some embodiments, Compound A, or a pharmaceutically acceptable salt thereof, is amorphous. In some embodiments, Compound A, or a pharmaceutically acceptable salt thereof, is in crystal form.

As used herein, the term “a metabolite of Compound A” refers to an intermediate or end product of Compound A after metabolism. In some embodiments, a metabolite of Compound A is a compound of formula:

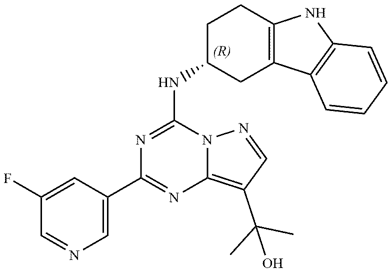 [SMILES: CC(C)(O)c1cnn2c(N[C@@H]3CCc4[nH]c5ccccc5c4C3)nc(-c3cncc(F)c3)nc12]

(Compound B), or a pharmaceutically acceptable salt thereof. In some embodiments, a metabolite of Compound A is a compound of formula:

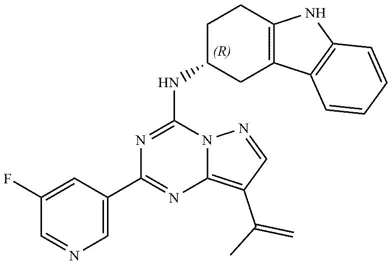 [SMILES: C=C(C)c1cnn2c(N[C@@H]3CCc4[nH]c5ccccc5c4C3)nc(-c3cncc(F)c3)nc12]

(Compound C), or a pharmaceutically acceptable salt thereof.

As used herein, the term “a prodrug thereof” refers to a compound, which produces the recited compound(s) after metabolism. In some embodiments, a prodrug of a metabolite of Compound A is a compound, which produces a metabolite of Compound A after metabolism. In some embodiments, a prodrug of a metabolite of Compound A is a compound, which produces Compound B, or a pharmaceutically acceptable salt thereof, after metabolism. In some embodiments, a prodrug of a metabolite of Compound A is a compound, which produces Compound C, or a pharmaceutically acceptable salt thereof, after metabolism.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

As used herein, the terms “about” or “approximately” have the meaning of within 20% of a given value or range. In some embodiments, the term “about” refers to within 20%, 19%, 18%, 17%, 16%, 15%, 14%, 13%, 12%, 11%, 10%, 9%, 8%, 7%, 6%, 5%, 4%, 3%, 2%, or 1% of a given value.

As used herein, the terms “increases,” “elevates,” or “enhances,” are used interchangeably and encompass any measurable increase in a biological function and/or biological activity and/or a concentration and/or amount, such as, for example, an increase in AHR nuclear positivity. For example, an increase can be by at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 100%, about 2-fold, about 3-fold, about 4-fold, about 5-fold, about 6-fold, about 7-fold, about 8-fold, about 9-fold, about 10-fold, about 20-fold, about 25-fold, about 50-fold, about 100-fold, or higher, relative to a control or baseline amount of a function, or activity, or concentration.

As used herein, the terms “increased concentration,” or “increased levels” or “increased amounts” of a substance (e.g., nuclear AHR) in a sample, such as a tumor biopsy, refers to an increase in the amount of the substance of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 100%, about 2-fold, about 3-fold, about 4-fold, about 5-fold, about 6-fold, about 7-fold, about 8-fold, about 9-fold, about 10-fold, about 20-fold, about 25-fold, about 50-fold, about 100-fold, or higher, relative to the amount of the substance in a control sample or control samples, such as an individual or group of individuals who are not suffering from the disease or disorder (e.g., cancer) or an internal control, as determined by techniques known in the art. A subject can also be determined to have an “increased concentration” or “increased amount” of a substance if the concentration of the substance is increased by one standard deviation, two standard deviations, three standard deviations, four standard deviations, five standard deviations, or more relative to the mean (average) or median amount of the substance in a control group of samples or a baseline group of samples or a retrospective analysis of patient samples. As practiced in the art, such control or baseline levels can be previously determined, or measured prior to the measurement in the sample, or can be obtained from a database of such control samples. In other words, the control and subject samples do not have to be tested simultaneously. Similarly, “reduced concentration,” “decreased concentrations,” “decreased amounts,” “lowered levels,” or “reduced levels” refers to a decrease in concentration or a decrease in level by at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or 100% in a sample relative to a control.

As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment or therapy.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the term “patient” refers to an animal, preferably a mammal, and, most preferably, a human.

As used herein, a patient or subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment or therapy.

As used herein, the term “a therapeutically effective amount of” refers to the amount of an AHR inhibitor (e.g., compound A, or a pharmaceutically acceptable salt thereof), which is effective to inhibit AHR activity in a biological sample or in a patient. In some embodiments, “a therapeutically effective amount of” refers to the amount of an AHR inhibitor (e.g., compound A, or a pharmaceutically acceptable salt thereof), which measurably blocks AHR from translocating from the cytoplasm to the nucleus in the presence of a ligand. In some embodiments, “a therapeutically effective amount of” refers to the amount of an AHR inhibitor (e.g., compound A, or a pharmaceutically acceptable salt thereof), which measurably displaces an endogenous ligand which binds to AHR in the nucleus.

The term “promote(s) cancer regression” means that administering an effective amount of the drug, alone or in combination with one or more additional anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

As used herein, the terms “therapeutic efficacy” or “responsiveness to treatment” or “therapeutic benefit” or “benefit from therapy” refer to an improvement in one or more of overall survival, progression-free survival, partial response, complete response, and overall response rate and can also include a reduction in cancer or tumor growth or size, a decrease in severity of disease symptoms, an increase in frequency and duration of disease symptom-free periods, or a prevention of impairment or disability due to the disease affliction.

### 3. Description of Exemplary Methods and Uses

In some aspects and embodiments, the present invention provides methods of identifying or selecting a cancer patient who is AHR nuclear positive and/or has an AHR gene amplification for treatment with an AHR antagonist. In some aspects and embodiments, the methods comprise identifying or selecting a cancer patient who is AHR nuclear positive. Such methods can comprise, for example, determining whether a patient is AHR nuclear positive using available methods know in the art, such as, for example, IHC staining. In some aspects and embodiments, the methods comprise identifying or selecting a cancer patient who has an AHR gene amplification. In some embodiments, the method further comprises administering an AHR antagonist to the patient who is AHR nuclear positive, such as Compound A or a pharmaceutically acceptable salt thereof. In some embodiments, the method further comprises administering an AHR antagonist to the patient who is AHR nuclear positive, such as a metabolite of Compound A, or a pharmaceutically acceptable salt thereof, or a prodrug thereof.

In some embodiments, the present invention provides a method for IHC staining a tumor tissue of a patient, comprising staining a tumor tissue section using an AHR monoclonal antibody. In some embodiments, an AHR monoclonal antibody is FF3399.

In some embodiments, a tumor tissue section is an about 4 µm thick tissue section on a positively charged glass slide. In some embodiments, a tumor tissue section is about 2.0, 2.5, 3.0, 3.5, 4.5, 5.0, 5.5, or 6.0 µm thick on a positively-charged glass slide. In some embodiments, a tumor tissue section is stained at about pH 6.0. In some embodiments, a tumor tissue section is stained at about pH 5.0, 5.5, 6.5, or 7.0. In some embodiments, a tumor tissue section is stained for about 40 minutes. In some embodiments, a tumor tissue section is stained for about 20, 25, 30, 35, 45, 50, 55, or 60 minutes.

In some embodiments, the present invention provides a method for identifying or selecting a cancer patient who is AHR nuclear positive, comprising IHC staining a tumor tissue of a patient, and selecting a patient who is AHR nuclear staining positive.

As used herein, the term “AHR nuclear positive” refers to that certain percentage of cells in a sample, such as a tumor sample, that have a detectable amount of AHR in nucleus. In some embodiments, AHR nuclear positive refers to that about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or 100% of cells in a sample have a detectable amount of AHR in the nucleus. As used herein, the term “AHR nuclear positive” refers to that certain percentage of cells in a tumor biopsy core have a detectable amount of AHR in nucleus. In some embodiments, AHR nuclear positive refers to that about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or 100% of cells in a tumor biopsy core have a detectable amount of AHR in nucleus. In some embodiments, AHR nuclear positive refers to that about 5% or more cells in a tumor biopsy core have a detectable amount of AHR in nucleus. In some embodiments, AHR nuclear positive refers to that about 20% or more of cells in a tumor biopsy core have a detectable amount of AHR in nucleus. In some embodiments, AHR nuclear positive refers to that about 50% or more of cells in a tumor biopsy core have a detectable amount of AHR in nucleus. In some embodiments, a tumor biopsy core refers to a tumor region of the tumor biopsy core. In some embodiments, a tumor biopsy core refers to a tumor microenvironment (or stroma) region of the tumor biopsy core.

In some embodiments, the present invention provides a method for identifying or selecting a cancer patient who has AHR gene amplification, comprising measuring AHR gene copies in a sample from the patient, such as a tumor sample, and selecting a patient who has AHR gene amplification for treatment with an AHR antagonist. In some embodiments, the method further comprises administering an AHR antagonist to the patient who has AHR nuclear gene amplification, such as Compound A or a pharmaceutically acceptable salt thereof. In some embodiments, the method further comprises administering an AHR antagonist to the patient who has AHR nuclear gene amplification, such as a metabolite of Compound A, or a pharmaceutically acceptable salt thereof, or a prodrug thereof.

As used herein, the term “AHR gene amplification” refers to that certain percentage of cells in a sample, such as a tumor sample, having a detectable amount of AHR gene amplification. In some embodiments, AHR gene amplification refers to that about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or 100% of cells, such as tumor cells, in a sample have at least about three copies of AHR, at least about four copies of AHR, at least about five copies of AHR, at least about six copies of AHR, at least about seven copies of AHR, at least about eight copies of AHR, at least about nine copies of AHR, at least about ten copies of AHR, at least about eleven copies of AHR, at least about twelve copies of AHR, at least about nine copies of AHR, at least about ten copies of AHR, at least about eleven copies of AHR, at least about twelve copies of AHR, at least about thirteen copies of AHR, at least about fourteen copies of AHR, at least about fifteen copies of AHR, at least about twenty copies of AHR, or more. In some embodiments, AHR gene amplification refers to that about 10% tumor cells in a sample have at least about 15 copies of AHR. In some embodiments, AHR gene amplification refers to that about 40% tumor cells in a sample have at least about 4 copies of AHR. In some embodiments, AHR gene amplification refers to that about 10% tumor cells in a sample have at least about four copies of AHR.

Methods and assays of measuring or determining AHR gene amplification and AHR overexpression in a sample, including overexpression of AHR in the nucleus or AHR nuclear staining positive, are known in the art and can be used with the methods described herein. There are a variety of methods to detect the amount of AHR translocating from the cytoplasm to the nucleus upon binding to a ligand. Non-limiting examples of such assays and methods include immunoassays, such as immunohistochemistry, next-generation sequencing (NGS), RNAscope, and fluorescent in situ hybridization (FISH).

In some embodiments, Next Generation Sequence (NGS) is used to detect AHR gene amplification or to detect an amount of AHR translocating from the cytoplasm to the nucleus upon binding to a ligand. Next Generation Sequencing (NGS) encompasses DNA sequencing using targeted panels, Whole exome sequencing, and whole genome sequencing are methods that allow determination of copy number variations (CNV) in genes of interest (Zhao, BMC bioinformatics 2012). Copy number alterations include deletions or amplifications of genes. To detect CNV, DNA is isolated from the samples of interest, which can be fresh or FFPE tissue, such as biopsies and blood, among other tissues. The DNA is amplified and labeled to form libraries which are then run into NGS sequencers. The results from the sequencers are then analyzed using computational algorithms specifically designed to infer CNVs.

In some embodiments, RNAscope is used to detect AHR gene amplification or to detect an amount of AHR translocating from the cytoplasm to the nucleus upon binding to a ligand. RNAscope is a method that allows for in situ RNA analysis detection and quantification in formalin-fixed, paraffin-embedded tissues (Wand J Mol Diagn. 2012). RNA ISH and particularly RNAscope can be utilized to quantify expression of a given gene in cells. For example, RNAscope was utilized herein to assess AHR mRNA expression in cancer cell lines and immune cells from 10 tumor types in a tumor microarray (Pancreas, Colon, Kidney, Head & Neck, Melanoma, Prostate, Lung, Ovary, Bladder and Breast). Images were scanned and analyzed using computational software (HALO). This method was suitable to determine AHR expression in tumor cells and tumor microenvironment by H-SCORE.

In some embodiments, fluorescent in situ hybridization (FISH) is used to detect AHR gene amplification or to detect an amount of AHR translocating from the cytoplasm to the nucleus upon binding to a ligand. For example, cells are obtained from a biological sample, such as an FFPE sample, and hybridized with a probe set specific for AHR. Probe signals are captured and inverted DAPI images reviewed. Samples can be deemed positive for AHR amplification if various criteria are met. For example, ≥10% tumor cells ≥ 15 copies of AHR, ≥ 40% tumor cells ≥ 4 copies of AHR, and/or ≥ 10% tumor cells ≥ 4 copies (cluster) of AHR.

In some embodiments, an immunohistochemistry (IHC) staining assay is used to detect an amount of AHR translocating from the cytoplasm to the nucleus upon binding to a ligand. In some embodiments, an immunohistochemistry (IHC) staining assay is used to detect AHR gene amplification. IHC is a method that uses antibodies to check for certain antigens (markers), such as AHR, in a sample of tissue. The antibodies are usually linked to an enzyme or a fluorescent dye. After the antibodies bind to the antigen in the tissue sample, the enzyme or dye is activated, and the antigen can then be seen under a microscope.

In some embodiments, an IHC staining assay is as described in Example 1 herein. Accordingly, in some embodiments, AHR nuclear positive refers to AHR nuclear staining positive in an IHC staining assay. In some embodiments, AHR nuclear staining positive refers to that a detectable number of cells in a tumor biopsy core are staining positive in an IHC staining assay. In some embodiments, AHR nuclear staining positive refers to that about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or 100% of cells in a tumor biopsy core are staining positive in an IHC staining assay.

In some embodiments, a tumor biopsy core refers to a tumor region of the tumor biopsy core. In some embodiments, a tumor biopsy core refers to a tumor microenvironment (or stroma) region of the tumor biopsy core.

In some embodiments, an IHC staining assay comprises measuring staining intensity in a tumor biopsy core. There are a variety of methods to measure staining intensity in an IHC staining assay. In some embodiments, staining intensity is measured by the methods as described in Example 1 herein. In some embodiments, staining intensity is measured by visual scoring, for example, by manual scoring using conventional light microscopy. In some embodiments, staining intensity is measured by computational tissue analysis (CTA) scoring. The staining intensity levels can be no staining (0), weak staining (1+), median staining (2+), or strong staining (3+). In some embodiments, staining positive refers to all staining intensity (including 1+, 2+, and 3+ intensities). In some embodiments, staining positive refers to pooled 2+ and 3+ staining intensity (including 2+ and 3+ intensities). In some embodiments, staining intensity is measured in a tumor region of a tumor biopsy core. In some embodiments, staining intensity is measured in a tumor microenvironment (or stroma) region of a tumor biopsy core.

As described herein, IHC staining has shown that the percentage of AHR nuclear positive patients varies significantly across different types of cancer. Accordingly, selecting AHR nuclear positive patients prior to an AHR inhibitor treatment can be particularly beneficial for certain types of cancer. In some embodiments, the present invention provides a method of selecting an AHR nuclear positive patient of a particular cancer type. In some embodiments, the particular cancer type is selected from bladder cancer, melanoma, ovarian cancer, and HNSCC.

In some embodiments, a method of selecting an AHR nuclear positive patient is for selecting patients of bladder cancer. In some embodiments, IHC staining shows that certain percent of bladder cancer is AHR nuclear positive, as shown in Tables 1 and 6 below. In some embodiments, IHC staining shows that about 58% of bladder cancer patients have about 5% or more cells that are AHR nuclear staining positive at all intensity (including 1+, 2+, and 3+ intensities) by CTA scoring in a tumor region of a tumor biopsy core. In some embodiments, IHC staining shows that about 46% of bladder cancer patients have about 20% or more cells that are AHR nuclear staining positive at all intensity by CTA scoring in a tumor region of a tumor biopsy core. In some embodiments, IHC staining shows that about 36% of bladder cancer patients have about 50% or more cells that are AHR nuclear staining positive at all intensity by CTA scoring in a tumor region of a tumor biopsy core. In some embodiments, IHC staining shows that about 51% of bladder cancer patients have about 5% or more cells that are AHR nuclear staining positive at all intensity by CTA scoring in a tumor microenvironment (or stroma) region of a tumor biopsy core. In some embodiments, IHC staining shows that about 36% of bladder cancer patients have about 20% or more cells that are AHR nuclear staining positive at all intensity by CTA scoring in a tumor microenvironment (or stroma) region of a tumor biopsy core. In some embodiments, IHC staining shows that about 10% of bladder cancer patients have about 50% or more cells that are AHR nuclear staining positive at all intensity by CTA scoring in a tumor microenvironment (or stroma) region of a tumor biopsy core.

In some embodiments, IHC staining shows that about 45% of bladder cancer patients have about 5% or more cells that are AHR nuclear staining positive at pooled 2+ and 3+ staining intensity by CTA scoring in a tumor region of a tumor biopsy core. In some embodiments, IHC staining shows that about 35% of bladder cancer patients have about 20% or more cells that are AHR nuclear staining positive at pooled 2+ and 3+ staining intensity by CTA scoring in a tumor region of a tumor biopsy core. In some embodiments, IHC staining shows that about 21% of bladder cancer patients have about 50% or more cells that are AHR nuclear staining positive at pooled 2+ and 3+ staining intensity by CTA scoring in a tumor region of a tumor biopsy core. In some embodiments, IHC staining shows that about 43% of bladder cancer patients have about 5% or more cells that are AHR nuclear staining positive at pooled 2+ and 3+ staining intensity by CTA scoring in a tumor microenvironment (or stroma) region of a tumor biopsy core. In some embodiments, IHC staining shows that about 20% of bladder cancer patients have about 20% or more cells that are AHR nuclear staining positive at pooled 2+ and 3+ staining intensity by CTA scoring in a tumor microenvironment (or stroma) region of a tumor biopsy core. In some embodiments, IHC staining shows that about 1% of bladder cancer patients have about 50% or more cells that are AHR nuclear staining positive at pooled 2+ and 3+ staining intensity by CTA scoring in a tumor microenvironment (or stroma) region of a tumor biopsy core.

In some embodiments, a method of selecting an AHR nuclear positive patient is for selecting patients of melanoma. In some embodiments, IHC staining shows that certain percent of melanoma is AHR nuclear positive, as shown in Tables 2, 3, 7, and 8 below. In some embodiments, IHC staining shows that about 11-20% of melanoma patients have about 5% or more cells that are AHR nuclear staining positive at all intensity by CTA scoring in a tumor region of a tumor biopsy core. In some embodiments, IHC staining shows that about 5-13% of melanoma patients have about 20% or more cells that are AHR nuclear staining positive at all intensity by CTA scoring in a tumor region of a tumor biopsy core. In some embodiments, IHC staining shows that about 1-3% of melanoma patients have about 50% or more cells that are AHR nuclear staining positive at all intensity by CTA scoring in a tumor region of a tumor biopsy core. In some embodiments, IHC staining shows that about 9-11% of melanoma patients have about 5% or more cells that are AHR nuclear staining positive at all intensity by CTA scoring in a tumor microenvironment (or stroma) region of a tumor biopsy core. In some embodiments, IHC staining shows that about 3-5% of melanoma patients have about 20% or more cells that are AHR nuclear staining positive at all intensity by CTA scoring in a tumor microenvironment (or stroma) region of a tumor biopsy core. In some embodiments, IHC staining shows that about 6-16% of melanoma patients have about 5% or more cells that are AHR nuclear staining positive at pooled 2+ and 3+ staining intensity by CTA scoring in a tumor region of a tumor biopsy core. In some embodiments, IHC staining shows that about 3-8% of melanoma patients have about 20% or more cells that are AHR nuclear staining positive at pooled 2+ and 3+ staining intensity by CTA scoring in a tumor region of a tumor biopsy core. In some embodiments, IHC staining shows that about 3% of melanoma patients have about 50% or more cells that are AHR nuclear staining positive at pooled 2+ and 3+ staining intensity by CTA scoring in a tumor region of a tumor biopsy core. In some embodiments, IHC staining shows that about 5-9% of melanoma patients have about 5% or more cells that are AHR nuclear staining positive at pooled 2+ and 3+ staining intensity by CTA scoring in a tumor microenvironment (or stroma) region of a tumor biopsy core. In some embodiments, IHC staining shows that about 3% of melanoma patients have about 20% or more cells that are AHR nuclear staining positive at pooled 2+ and 3+ staining intensity by CTA scoring in a tumor microenvironment (or stroma) region of a tumor biopsy core.

In some embodiments, a method of selecting an AHR nuclear positive patient is for selecting patients of ovarian cancer. In some embodiments, IHC staining shows that certain percent of ovarian cancer is AHR nuclear positive, as shown in Tables 4 and 9 below. In some embodiments, IHC staining shows that about 10% of ovarian cancer patients have about 5% or more cells that are AHR nuclear staining positive at all intensity by CTA scoring in a tumor region of a tumor biopsy core. In some embodiments, IHC staining shows that about 10% of ovarian cancer patients have about 5% or more cells that are AHR nuclear staining positive at all intensity by CTA scoring in a tumor microenvironment (or stroma) region of a tumor biopsy core. In some embodiments, IHC staining shows that about 3% of ovarian cancer patients have about 20% or more cells that are AHR nuclear staining positive at all intensity by CTA scoring in a tumor microenvironment (or stroma) region of a tumor biopsy core. In some embodiments, IHC staining shows that about 6% of ovarian cancer patients have about 5% or more cells that are AHR nuclear staining positive at pooled 2+ and 3+ staining intensity by CTA scoring in a tumor region of a tumor biopsy core. In some embodiments, IHC staining shows that about 3% of ovarian cancer patients have about 5% or more cells that are AHR nuclear staining positive at pooled 2+ and 3+ staining intensity by CTA scoring in a tumor microenvironment (or stroma) region of a tumor biopsy core. In some embodiments, IHC staining shows that about 3% of ovarian cancer patients have about 20% or more cells that are AHR nuclear staining positive at pooled 2+ and 3+ staining intensity by CTA scoring in a tumor microenvironment (or stroma) region of a tumor biopsy core.

In some embodiments, a method of selecting an AHR nuclear positive patient is for selecting patients of HNSCC. In some embodiments, IHC staining shows that certain percent of HNSCC is AHR nuclear positive, as shown in Tables 5 and 10 below. In some embodiments, IHC staining shows that about 28% of HNSCC patients have about 5% or more cells that are AHR nuclear staining positive at all intensity by CTA scoring in a tumor region of a tumor biopsy core. In some embodiments, IHC staining shows that about 21% of HNSCC patients have about 20% or more cells that are AHR nuclear staining positive at all intensity by CTA scoring in a tumor region of a tumor biopsy core. In some embodiments, IHC staining shows that about 8% of HNSCC patients have about 50% or more cells that are AHR nuclear staining positive at all intensity by CTA scoring in a tumor region of a tumor biopsy core. In some embodiments, IHC staining shows that about 29% of HNSCC patients have about 5% or more cells that are AHR nuclear staining positive at all intensity by CTA scoring in a tumor microenvironment (or stroma) region of a tumor biopsy core. In some embodiments, IHC staining shows that about 13% of HNSCC patients have about 20% or more cells that are AHR nuclear staining positive at all intensity by CTA scoring in a tumor microenvironment (or stroma) region of a tumor biopsy core. In some embodiments, IHC staining shows that about 1% of HNSCC patients have about 50% or more cells that are AHR nuclear staining positive at all intensity by CTA scoring in a tumor microenvironment (or stroma) region of a tumor biopsy core. In some embodiments, IHC staining shows that about 25% of HNSCC patients have about 5% or more cells that are AHR nuclear staining positive at pooled 2+ and 3+ staining intensity by CTA scoring in a tumor region of a tumor biopsy core. In some embodiments, IHC staining shows that about 13% of HNSCC patients have about 20% or more cells that are AHR nuclear staining positive at pooled 2+ and 3+ staining intensity by CTA scoring in a tumor region of a tumor biopsy core. In some embodiments, IHC staining shows that about 3% of HNSCC patients have about 50% or more cells that are AHR nuclear staining positive at pooled 2+ and 3+ staining intensity by CTA scoring in a tumor region of a tumor biopsy core. In some embodiments, IHC staining shows that about 21% of HNSCC patients have about 5% or more cells that are AHR nuclear staining positive at pooled 2+ and 3+ staining intensity by CTA scoring in a tumor microenvironment (or stroma) region of a tumor biopsy core. In some embodiments, IHC staining shows that about 4% of HNSCC patients have about 20% or more cells that are AHR nuclear staining positive at pooled 2+ and 3+ staining intensity by CTA scoring in a tumor microenvironment (or stroma) region of a tumor biopsy core.

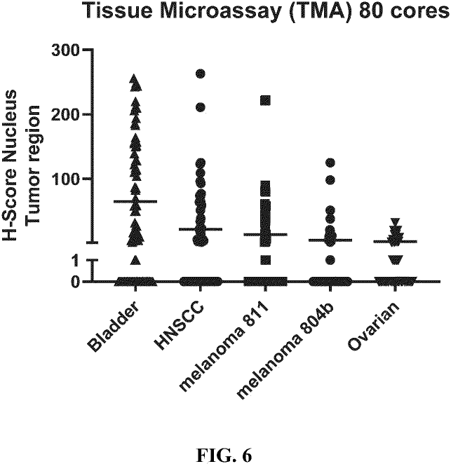

In some embodiments, the present invention provides a method of selecting an AHR nuclear positive patient comprising selecting a cancer patient having an H-Score equal to or higher than the mean value of the cancer type. In some embodiments, H-scores and mean values from IHC staining for bladder cancer, melanoma, ovarian cancer, and HNSCC are as shown in FIG. 6. In some embodiments, the present invention provides a method of selecting an AHR nuclear positive patient comprising selecting a patient having an H-score equal to or higher than the mean H-score of bladder cancer as shown in FIG. 6. In some embodiments, the present invention provides a method of selecting an AHR nuclear positive patient comprising selecting a patient having an H-score equal to or higher than the mean H-score of melanoma as shown in FIG. 6. In some embodiments, the present invention provides a method of selecting an AHR nuclear positive patient comprising selecting a patient having an H-score equal to or higher than the mean H-score of ovarian cancer as shown in FIG. 6. In some embodiments, the present invention provides a method of selecting an AHR nuclear positive patient comprising selecting a patient having an H-score equal to or higher than the mean H-score of HNSCC as shown in FIG. 6.In some embodiments, the present invention provides a method of treating cancer in a patient, comprising selecting a patient who is AHR nuclear positive, and administering to the patient a therapeutically effective amount of an AHR inhibitor, or a pharmaceutical composition thereof. In some embodiments, a cancer is bladder cancer. In some embodiments, a bladder cancer is transitional cell carcinoma (TCC). In some embodiments, a cancer is melanoma. In some embodiments, a cancer is ovarian cancer. In some embodiments, a cancer is HNSCC.

In some embodiments, the present invention provides a method of treating cancer in a patient, comprising selecting a patient who is AHR nuclear positive by an IHC staining assay, as described herein, and administering to the patient a therapeutically effective amount of an AHR inhibitor, as described herein, or a pharmaceutical composition thereof.

In some embodiments, the present invention provides a method of treating cancer in a patient, comprising selecting a patient having about 5% or more cells that are AHR nuclear staining positive at all intensity or at pooled 2+ and 3+ staining intensity by CTA scoring in a tumor region of a tumor biopsy core, and administering to the patient a therapeutically effective amount of an AHR inhibitor, as described herein, or a pharmaceutical composition thereof.

In some embodiments, the present invention provides a method of treating cancer in a patient, comprising selecting a patient having about 20% or more cells that are AHR nuclear staining positive at all intensity or at pooled 2+ and 3+ staining intensity by CTA scoring in a tumor region of a tumor biopsy core, and administering to the patient a therapeutically effective amount of an AHR inhibitor, as described herein, or a pharmaceutical composition thereof.

In some embodiments, the present invention provides a method of treating cancer in a patient, comprising selecting a patient having about 50% or more cells that are AHR nuclear staining positive at all intensity or at pooled 2+ and 3+ staining intensity by CTA scoring in a tumor region of a tumor biopsy core, and administering to the patient a therapeutically effective amount of an AHR inhibitor, as described herein, or a pharmaceutical composition thereof.

In some embodiments, an AHR inhibitor is selected from the compounds as described in WO2017202816A1, WO2018085348A1, WO2018195397, WO2019101642A1, WO2019101643A1, WO2019101641A1, WO2019101647A1, WO2019036657A1, US10570138B2, US10689388B1, US10696650B2, WO2020051207A2, WO2020081636A1, and WO2020081840A1.

In some embodiments, an AHR inhibitor is selected from the compounds as described in WO2018195397, US20180327411, WO2019036657, and WO2020081636A1, the contents of each of which are incorporated herein by reference in their entirety.

In some embodiments, an AHR inhibitor is selected from the compounds as described in WO2018195397, US20180327411, and PCT/US2019/056455, the contents of each of which are incorporated herein by reference in their entirety.

In some embodiments, an AHR inhibitor is Compound A, or a pharmaceutically acceptable salt thereof. In some embodiments, an AHR inhibitor is a metabolite of Compound A, or a pharmaceutically acceptable salt thereof, or a prodrug thereof. In some embodiments, an AHR inhibitor is Compound B, or a pharmaceutically acceptable salt thereof, or a prodrug thereof. In some embodiments, an AHR inhibitor is Compound C, or a pharmaceutically acceptable salt thereof, or a prodrug thereof.

In some embodiments, the present invention provides a method of treating bladder cancer in a patient, comprising:
IHC staining a tumor tissue of a patient;
selecting a patient having about 5% or more cells in a tumor region of a tumor biopsy core which are AHR nuclear staining positive at all staining intensity (including 1+, 2+, and 3+ intensities) or at pooled 2+ and 3+ staining intensity by CTA scoring; and
administering to the patient a therapeutically effective amount of Compound A, or a pharmaceutically acceptable salt thereof.

In some embodiments, the present invention provides a method of treating bladder cancer in a patient, comprising:
IHC staining a tumor tissue of a patient;
selecting a patient having about 20% or more cells in a tumor region of a tumor biopsy core which are AHR nuclear staining positive at all staining intensity (including 1+, 2+, and 3+ intensities) or at pooled 2+ and 3+ staining intensity by CTA scoring; and
administering to the patient a therapeutically effective amount of Compound A, or a pharmaceutically acceptable salt thereof.

In some embodiments, the present invention provides a method of treating bladder cancer in a patient, comprising:
IHC staining a tumor tissue of a patient;
selecting a patient having about 50% or more cells in a tumor region of a tumor biopsy core which are AHR nuclear staining positive at all staining intensity (including 1+, 2+, and 3+ intensities) or at pooled 2+ and 3+ staining intensity by CTA scoring; and
administering to the patient a therapeutically effective amount of Compound A, or a pharmaceutically acceptable salt thereof.

In some embodiments, the present invention provides a method of treating HNSCC in a patient, comprising:
IHC staining a tumor tissue of a patient;
selecting a patient having about 5% or more cells in a tumor region of a tumor biopsy core which are AHR nuclear staining positive at all staining intensity (including 1+, 2+, and 3+ intensities) or at pooled 2+ and 3+ staining intensity by CTA scoring; and
administering to the patient a therapeutically effective amount of Compound A, or a pharmaceutically acceptable salt thereof.

In some embodiments, the present invention provides a method of treating HNSCC in a patient, comprising:
IHC staining a tumor tissue of a patient;
selecting a patient having about 20% or more cells in a tumor region of a tumor biopsy core which are AHR nuclear staining positive at all staining intensity (including 1+, 2+, and 3+ intensities) or at pooled 2+ and 3+ staining intensity by CTA scoring; and
administering to the patient a therapeutically effective amount of Compound A, or a pharmaceutically acceptable salt thereof.

In some embodiments, the present invention provides a method of treating HNSCC in a patient, comprising:
IHC staining a tumor tissue of a patient;
selecting a patient having about 50% or more cells in a tumor region of a tumor biopsy core which are AHR nuclear staining positive at all staining intensity (including 1+, 2+, and 3+ intensities) or at pooled 2+ and 3+ staining intensity by CTA scoring; and
administering to the patient a therapeutically effective amount of Compound A, or a pharmaceutically acceptable salt thereof.

In some embodiments, the present invention provides a method of treating ovarian cancer in a patient, comprising:
IHC staining a tumor tissue of a patient;
selecting a patient having about 5% or more cells in a tumor region of a tumor biopsy core which are AHR nuclear staining positive at all staining intensity (including 1+, 2+, and 3+ intensities) or at pooled 2+ and 3+ staining intensity by CTA scoring; and
administering to the patient a therapeutically effective amount of Compound A, or a pharmaceutically acceptable salt thereof.

In some embodiments, the present invention provides a method of treating ovarian cancer in a patient, comprising:
IHC staining a tumor tissue of a patient;
selecting a patient having about 20% or more cells in a tumor region of a tumor biopsy core which are AHR nuclear staining positive at all staining intensity (including 1+, 2+, and 3+ intensities) or at pooled 2+ and 3+ staining intensity by CTA scoring; and
administering to the patient a therapeutically effective amount of Compound A, or a pharmaceutically acceptable salt thereof.

In some embodiments, the present invention provides a method of treating ovarian cancer in a patient, comprising:
IHC staining a tumor tissue of a patient;
selecting a patient having about 50% or more cells in a tumor region of a tumor biopsy core which are AHR nuclear staining positive at all staining intensity (including 1+, 2+, and 3+ intensities) or at pooled 2+ and 3+ staining intensity by CTA scoring; and
administering to the patient a therapeutically effective amount of Compound A, or a pharmaceutically acceptable salt thereof.

In some embodiments, the present invention provides a method of treating melanoma in a patient, comprising:
IHC staining a tumor tissue of a patient;
selecting a patient having about 5% or more cells in a tumor region of a tumor biopsy core which are AHR nuclear staining positive at all staining intensity (including 1+, 2+, and 3+ intensities) or at pooled 2+ and 3+ staining intensity by CTA scoring; and
administering to the patient a therapeutically effective amount of Compound A, or a pharmaceutically acceptable salt thereof.

In some embodiments, the present invention provides a method of treating melanoma in a patient, comprising:
IHC staining a tumor tissue of a patient;
selecting a patient having about 20% or more cells in a tumor region of a tumor biopsy core which are AHR nuclear staining positive at all staining intensity (including 1+, 2+, and 3+ intensities) or at pooled 2+ and 3+ staining intensity by CTA scoring; and
administering to the patient a therapeutically effective amount of Compound A, or a pharmaceutically acceptable salt thereof.

In some embodiments, the present invention provides a method of treating melanoma in a patient, comprising:
IHC staining a tumor tissue of a patient;
selecting a patient having about 50% or more cells in a tumor region of a tumor biopsy core which are AHR nuclear staining positive at all staining intensity (including 1+, 2+, and 3+ intensities) or at pooled 2+ and 3+ staining intensity by CTA scoring; and
administering to the patient a therapeutically effective amount of Compound A, or a pharmaceutically acceptable salt thereof.

### 4. Formulation and Administration

In some embodiments, a method described herein comprises administering a pharmaceutical composition comprising an AHR inhibitor, as described herein, and a pharmaceutically acceptable carrier, adjuvant, or vehicle. In some embodiments, the amount of an AHR inhibitor in a composition is such that is effective to measurably block AHR from translocating from the cytoplasm to the nucleus in the presence of a ligand in a biological sample or in a patient. In some embodiments, an AHR inhibitor composition is formulated for oral administration to a patient.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer’s solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01 - 100 mg/kg body weight/day of the inhibitor can be administered to a patient receiving these compositions.It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

In some embodiments, a method of the present invention comprises administering daily to a patient about 100 - 2000 mg of Compound A, or a pharmaceutically acceptable salt thereof. In some embodiments, a method of the present invention comprises administering daily to a patient about 150 - 1800 mg of Compound A, or a pharmaceutically acceptable salt thereof. In some embodiments, a method of the present invention comprises administering daily to a patient about 200 - 1600 mg of Compound A, or a pharmaceutically acceptable salt thereof.

In some embodiments, a method of the present invention comprises administering daily to a patient about 200 mg of Compound A, or a pharmaceutically acceptable salt thereof. In some embodiments, a method of the present invention comprises administering daily to a patient about 400 mg of Compound A, or a pharmaceutically acceptable salt thereof. In some embodiments, a method of the present invention comprises administering daily to a patient about 600 mg of Compound A, or a pharmaceutically acceptable salt thereof. In some embodiments, a method of the present invention comprises administering daily to a patient about 800 mg of Compound A, or a pharmaceutically acceptable salt thereof. In some embodiments, a method of the present invention comprises administering daily to a patient about 1000 mg of Compound A, or a pharmaceutically acceptable salt thereof. In some embodiments, a method of the present invention comprises administering daily to a patient about 1200 mg of Compound A, or a pharmaceutically acceptable salt thereof. In some embodiments, a method of the present invention comprises administering daily to a patient about 1400 mg of Compound A, or a pharmaceutically acceptable salt thereof. In some embodiments, a method of the present invention comprises administering daily to a patient about 1600 mg of Compound A, or a pharmaceutically acceptable salt thereof. In some embodiments, a method of the present invention comprises administering a formulation or a unit dosage form of Compound A once daily. In some embodiments, a method of the present invention comprises administering a formulation or a unit dosage form of Compound A twice daily. In some embodiments, a method of the present invention comprises administering a formulation or a unit dosage form of Compound A three times daily. In some embodiments, a method of the present invention comprises administering a formulation or a unit dosage form of Compound A four times daily.

In some embodiments, where the patient is administered daily about 1200 mg of Compound A, or a pharmaceutically acceptable salt thereof, the dosing is twice daily or BID, i.e., two separate about 600 mg doses. In some embodiments, where the patient is administered daily about 1200 mg of Compound A, or a pharmaceutically acceptable salt thereof, the dosing is thrice daily or TID, i.e., three separate about 400 mg doses. In some embodiments, where the patient is administered daily about 1200 mg of Compound A, or a pharmaceutically acceptable salt thereof, the dosing is four-times daily or QID, i.e., four separate about 300 mg doses.

In some embodiments, where the patient is administered daily about 1600 mg of Compound A, or a pharmaceutically acceptable salt thereof, the dosing is twice daily or BID, i.e., two separate about 800 mg doses. In some embodiments, where the patient is administered daily about 1600 mg of Compound A, or a pharmaceutically acceptable salt thereof, the dosing is thrice daily or TID, i.e., three separate about 533 mg doses. In some embodiments, where the patient is administered daily about 1600 mg of Compound A, or a pharmaceutically acceptable salt thereof, the dosing is four-times daily or QID, i.e., four separate about 400 mg doses.

### 5. Uses

In some aspects and embodiments, the present invention provides a method of treating a proliferative disorder, such as cancer, in a patient, comprising selecting a patient who is AHR nuclear positive, for example, using a method as described herein, and administering to the patient a therapeutically effective amount of an AHR antagonist, for example, as described herein. In some embodiments, a treatment method further comprises measuring or determining whether a tumor sample from the patient is AHR nuclear positive, for example, using an IHC staining method as described herein.

In some aspects and embodiments, the present invention provides a method of treating a proliferative disorder, such as cancer, in a patient, comprising selecting a patient who has an AHR gene amplification, for example, using a method as described herein, and administering to the patient a therapeutically effective amount of an AHR antagonist, for example, as described herein. In some embodiments, a treatment method further comprises measuring or determining whether a tumor sample from the patient has an AHR gene amplification, for example, using any of the methods as described herein, for example, NGS, RNAscope, or FISH.

In some aspects and embodiments, the present invention provides a method of treating a proliferative disorder, such as cancer, in a patient who is AHR nuclear positive, comprising administering to the patient a therapeutically effective amount of an AHR antagonist, for example, as described herein. In some embodiments, the patient is determined as being AHR nuclear positive, for example, using a method as described herein. In some embodiments, a treatment method further comprises measuring or determining whether a tumor sample from the patient is AHR nuclear positive, for example, using an IHC staining method as described herein.

In some aspects and embodiments, the present invention provides a method of treating a proliferative disorder, such as cancer, in a patient who has an AHR gene amplification, comprising administering to the patient a therapeutically effective amount of an AHR antagonist, for example, as described herein. In some embodiments, the patient is determined as having an AHR gene amplification, for example, using a method as described herein. In some embodiments, a treatment method further comprises measuring or determining whether a tumor sample from the patient has an AHR gene amplification, for example, using any of the methods as described herein, for example, NGS, RNAscope, or FISH.

In some embodiments of these methods, the AHR antagonist is Compound A or a pharmaceutically acceptable salt thereof. In some embodiments of these methods, the AHR antagonist is a metabolite of Compound A, or a pharmaceutically acceptable salt thereof, or a prodrug thereof. In some embodiments, a metabolite of Compound A is Compound B or Compound C.

### CANCER

The cancer or proliferative disorder or tumor to be treated using the methods and uses described herein include, but are not limited to, a hematological cancer, a lymphoma, a myeloma, a leukemia, a neurological cancer, skin cancer, breast cancer, a prostate cancer, a colorectal cancer, lung cancer, head and neck cancer, a gastrointestinal cancer, a liver cancer, a pancreatic cancer, a genitourinary cancer, a bone cancer, renal cancer, and a vascular cancer.

In some embodiments, a cancer to be treated using the methods described herein can be selected from selected from bladder cancer, melanoma, ovarian cancer, and HNSCC.

A cancer to be treated using the methods described herein can be selected from colorectal cancer, such as microsatellite-stable (MSS) metastatic colorectal cancer, including advanced or progressive microsatellite-stable (MSS) CRC; non-small cell lung cancer (NSCLC), such as advanced and/or metastatic NSCLC; ovarian cancer; breast cancer, such as inflammatory breast cancer; endometrial cancer; cervical cancer; head and neck cancer; gastric cancer; gastroesophageal junction cancer; and bladder cancer. In some embodiments, a cancer is colorectal cancer. In some embodiments, the colorectal cancer is metastatic colorectal cancer. In some embodiments, the colorectal cancer is microsatellite-stable (MSS) metastatic colorectal cancer. In some embodiments, a cancer is advanced or progressive microsatellite-stable (MSS) CRC. In some embodiments, a cancer is non-small cell lung cancer (NSCLC). In some embodiments, a cancer is advanced and/or metastatic NSCLC. In some embodiments, a cancer is ovarian cancer. In some embodiments, a cancer is breast cancer. In some embodiments, a cancer is inflammatory breast cancer. In some embodiments, a cancer is endometrial cancer. In some embodiments, a cancer is endometrial cancer. In some embodiments, a cancer is head and neck cancer. In some embodiments, a cancer is gastric cancer. In some embodiments, a cancer is gastroesophageal junction cancer. In some embodiments, a cancer is bladder cancer.

In some embodiments, a cancer to be treated using the methods described herein can be selected from selected from bladder cancer, melanoma, ovarian cancer, and HNSCC.

Cancer includes, in some embodiments, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin’s disease or non-Hodgkin’s disease), Waldenstrom’s macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing’s tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm’s tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g. Grade I -Pilocytic Astrocytoma, Grade II - Low-grade Astrocytoma, Grade III - Anaplastic Astrocytoma, or Grade IV - Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor. In some embodiments, the patient is an adult human. In some embodiments, the patient is a child or pediatric patient.

Cancer includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin’s Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins’s lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the cancer is selected from hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom’s macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom’s macroglobulinemia, or medulloblastoma.

In some embodiments, the cancer is a solid tumor, such as a sarcoma, carcinoma, or lymphoma. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom’s macroglobulinemia; or medulloblastoma.

In some embodiments, the cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom’s macroglobulinemia, or medulloblastoma.

In some embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom’s macroglobulinemia, or medulloblastoma.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom’s macroglobulinemia. In some embodiments, the cancer is medulloblastoma.

In some embodiments, the cancer is Acute Lymphoblastic Leukemia (ALL), Acute Myeloid Leukemia (AML), Adrenocortical Carcinoma, Anal Cancer, Appendix Cancer, Atypical Teratoid/Rhabdoid Tumor, Basal Cell Carcinoma, Bile Duct Cancer, Bladder Cancer, Bone Cancer, Brain Tumor, Astrocytoma, Brain and Spinal Cord Tumor, Brain Stem Glioma, Central Nervous System Atypical Teratoid/Rhabdoid Tumor, Central Nervous System Embryonal Tumors, Breast Cancer, Bronchial Tumors, Burkitt Lymphoma, Carcinoid Tumor, Carcinoma of Unknown Primary, Central Nervous System Cancer, Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma, Ductal Carcinoma In Situ (DCIS), Embryonal Tumors, Endometrial Cancer, Ependymoblastoma, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Fibrous Histiocytoma of Bone, Gallbladder Cancer, Gastric Cancer, Gastrointestinal Carcinoid Tumor, Gastrointestinal Stromal Tumors (GIST), Germ Cell Tumor, Ovarian Germ Cell Tumor, Gestational Trophoblastic Tumor, Glioma, Hairy Cell Leukemia, Head and Neck Cancer, Heart Cancer, Hepatocellular Cancer, Histiocytosis, Langerhans Cell Cancer, Hodgkin Lymphoma, Hypopharyngeal Cancer, Intraocular Melanoma, Islet Cell Tumors, Kaposi Sarcoma, Kidney Cancer, Langerhans Cell Histiocytosis, Laryngeal Cancer, Leukemia, Lip and Oral Cavity Cancer, Liver Cancer, Lobular Carcinoma In Situ (LCIS), Lung Cancer, Lymphoma, AIDS-Related Lymphoma, Macroglobulinemia, Male Breast Cancer, Medulloblastoma, Medulloepithelioma, Melanoma, Merkel Cell Carcinoma, Malignant Mesothelioma, Metastatic Squamous Neck Cancer with Occult Primary, Midline Tract Carcinoma Involving NUT Gene, Mouth Cancer, Multiple Endocrine Neoplasia Syndrome, Multiple Myeloma/Plasma Cell Neoplasm, Mycosis Fungoides, Myelodysplastic Syndrome, Myelodysplastic/Myeloproliferative Neoplasm, Chronic Myelogenous Leukemia (CML), Acute Myeloid Leukemia (AML), Myeloma, Multiple Myeloma, Chronic Myeloproliferative Disorder, Nasal Cavity Cancer, Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip Cancer, Oropharyngeal Cancer, Osteosarcoma, Ovarian Cancer, Pancreatic Cancer, Papillomatosis, Paraganglioma, Paranasal Sinus Cancer, Nasal Cavity Cancer, Parathyroid Cancer, Penile Cancer, Pharyngeal Cancer, Pheochromocytoma, Pineal Parenchymal Tumors of Intermediate Differentiation, Pineoblastoma, Pituitary Tumor, Plasma Cell Neoplasm, Pleuropulmonary Blastoma, Breast Cancer, Primary Central Nervous System (CNS) Lymphoma, Prostate Cancer, Rectal Cancer, Renal Cell Cancer, Clear cell renal cell carcinoma, Renal Pelvis Cancer, Ureter Cancer, Transitional Cell Cancer, Retinoblastoma, Rhabdomyosarcoma, Salivary Gland Cancer, Sarcoma, Sezary Syndrome, Skin Cancer, Small Cell Lung Cancer, Small Intestine Cancer, Soft Tissue Sarcoma, Squamous Cell Carcinoma, Squamous Neck Cancer with Occult Primary, Squamous Cell Carcinoma of the Head and Neck (HNSCC), Stomach Cancer, Supratentorial Primitive Neuroectodermal Tumors, T-Cell Lymphoma, Testicular Cancer, Throat Cancer, Thymoma, Thymic Carcinoma, Thyroid Cancer, Transitional Cell Cancer of the Renal Pelvis and Ureter, Triple Negative Breast Cancer (TNBC), Gestational Trophoblastic Tumor, Unknown Primary, Unusual Cancer of Childhood, Urethral Cancer, Uterine Cancer, Uterine Sarcoma, Waldenstrom Macroglobulinemia, or Wilms Tumor.

In certain embodiments, the cancer is selected from bladder cancer, breast cancer (including TNBC), cervical cancer, colorectal cancer, chronic lymphocytic leukemia (CLL), diffuse large B-cell lymphoma (DLBCL), esophageal adenocarcinoma, glioblastoma, head and neck cancer, leukemia (acute and chronic), low-grade glioma, lung cancer (including adenocarcinoma, non-small cell lung cancer, and squamous cell carcinoma), Hodgkin’s lymphoma, non-Hodgkin lymphoma (NHL), melanoma, multiple myeloma (MM), ovarian cancer, pancreatic cancer, prostate cancer, renal cancer (including renal clear cell carcinoma and kidney papillary cell carcinoma), and stomach cancer.

In some embodiments, the cancer is small cell lung cancer, non-small cell lung cancer, colorectal cancer, multiple myeloma, acute myeloid leukemia (AML), acute lymphoblastic leukemia (ALL), pancreatic cancer, liver cancer, hepatocellular cancer, neuroblastoma, other solid tumors or other hematological cancers.

In some embodiments, the cancer is small cell lung cancer, non-small cell lung cancer, colorectal cancer, multiple myeloma, or AML.

The present invention further features methods and compositions for the diagnosis, prognosis and treatment of viral-associated cancers, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells (See https://clinicaltrials.gov/ct2/show/study/ NCT02631746); as well as virus-associated tumors in gastric cancer, nasopharyngeal carcinoma, cervical cancer, vaginal cancer, vulvar cancer, squamous cell carcinoma of the head and neck, and Merkel cell carcinoma. (See https://clinicaltrials.gov/ct2/show/study/NCT02488759; see also https://clinicaltrials.gov/ct2/show/study/NCT0240886; https://clinicaltrials.gov/ct2/show/ NCT02426892)

In some embodiments, the methods or uses described herein inhibit or reduce or arrest the growth or spread of a cancer or tumor. In some embodiments, the methods or uses described herein inhibit or reduce or arrest further growth of the cancer or tumor. In some embodiments, the methods or uses described herein reduce the size (e.g., volume or mass) of the cancer or tumor by at least 5%, at least 10%, at least 25%, at least 50%, at least 75%, at least 90% or at least 99% relative to the size of the cancer or tumor prior to treatment. In some embodiments, the methods or uses described herein reduce the quantity of the cancers or tumors in the patient by at least 5%, at least 10%, at least 25%, at least 50%, at least 75%, at least 90% or at least 99% relative to the quantity of cancers or tumors prior to treatment.

The compounds and compositions, according to the methods of the present invention, can be administered using any amount and any route of administration effective for treating or lessening the severity of a cancer or tumor. The exact amount required varies from subject to subject, depending on the species, age, and general condition of the subject, the severity of the disease or condition, the particular agent, its mode of administration, and the like. The compounds and compositions, according to the methods of the present invention, are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions is decided by the attending physician within the scope of sound medical judgment. The specific effective dose level for any particular patient or organism depends upon a variety of factors, including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The terms “patient” or “subject,” as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the disease or disorder being treated. In certain embodiments, the compounds of the invention can be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect. The following examples are provided for illustrative purposes only and are not to be construed as limiting this invention in any manner.

### EXEMPLIFICATION

Compound A can be prepared by methods known to one of ordinary skill in the art, for example, as described in WO2018195397 and US20180327411, the contents of which are incorporated herein by reference in their entirety.

Abbreviations:
CTA: computational tissue analysis.
TME: tumor microenvironment, or stroma region, which is a separate region from tumor region
TMA: tissue microarray. In Example 1, all TMAs are human tumor biopsies.
Staining intensity
   1+: weak staining
   2+: median staining
   3+: strong staining
All intensity: including 1+, 2+ and 3+ intensities
Pooled 2+ 3+ intensity: including 2+ and 3+ intensities
H score calculated from “% cells” positive for staining and the staining intensity: [((0 × (% cells at 0)) + ((1 × (% cells at 1+)) + ((2 × (% cells at 2+)) + ((3 × (% cells at 3))]
% cells positive per core: the percentage of cells that are AHR nuclear staining positive in a biopsy core
# of TME+ cores: the number of cores that have >50% (or 20%, 5%) AHR nuclear positive cells at all intensity in TME region
# of Tumor+ cores: the number of cores that have >50% (or 20%, 5%) AHR nuclear positive cells at all intensity in tumor region
# of total cores in the TMA: how many cores in the TMA (tumor microarray)
% TME+ cores: the percentage of cores that are >50% (or 20%, 5%) AHR nuclear positive in TME region
% Tumor+ cores: the percentage of cores that are >50% (or 20%, 5%) AHR nuclear positive in tumor region
% 1+ Nucleus: the percentage of positive cells with AHR nuclear staining at 1+ intensity in one biopsy core
% 2+ Nucleus: the percentage of positive cells with AHR nuclear staining at 2+ intensity in one biopsy core
% 3+ Nucleus: the percentage of positive cells with AHR nuclear staining at 3+ intensity in one biopsy core

### Example 1. IHC Staining Protocol: AHR Monoplex for Use in FFPE

Formalin-Fixed Paraffin Embedded (FFPE) tissue blocks of bladder carcinoma were sectioned into 4 µm-thick tissue sections onto positively-charged glass slides. The slides were stained using the Leica Bond RX autostainer platform with the Aryl Hydrocarbon Receptor (AHR) monoclonal antibody FF3399. Staining conditions were pH 6 for 40 minutes, DAB for 10 minutes. The Leica BPRD kit utilizes a goat anti rabbit polymer and a mouse anti-rabbit linker.

The antibody was applied to the tissue sections at a final concentration of 0.5 micrograms/milliliter; Isotype and concentration matched irrelevant antibody was used as a negative control. Each antibody run included two sections of normal human bladder as positive control as strong AHR staining is observed in the bladder transitional epithelium.

IHC stained glass slides were interpreted by use of manual scoring by a board-certified MD pathologist using conventional light microscopy. The intensity of staining for both nucleus and cytoplasm were graded on 0-3 scale according to the following criteria: 0 (no staining observed), 1 (weak staining), 2 (moderate staining) and 3 (strong staining). The frequency of each staining intensity was determined, and the results were reported using an H score according to the formula below: [((0 × (% cells at 0)) + ((1 × (% cells at 1+)) + ((2 × (% cells at 2+)) + ((3 × (% cells at 3))]

Alternatively samples were digitally analyzed (CTA scoring) using Flagship’s image analysis services via the Flotilla platform. Algorithms were applied which characterize each cell in a whole slide scan and generate numerous measured features about each cell, such as morphology or IHC stain-related measurements. Algorithms which further define the Tumor and Stroma will additionally be implemented to provide contextual data relevant to Immune Oncology research. Whole tissue slide or individual tumor cores on a tissue microarray (TMA) slide and Tumor/Stroma/Margin specific measurements of AHR expression were investigated. AHR scoring paradigms were digitally assessed. Similar to manual scoring, the intensity of staining for both nucleus and cytoplasm were graded on 0-3 scale according to the following criteria: 0 (no staining observed), 1 (weak staining), 2 (moderate staining) and 3 (strong staining). The frequency of each staining intensity was determined, and the results were reported using an H score according to the formula below: [((0 × (% cells at 0)) + ((1 × (% cells at 1+)) + ((2 × (% cells at 2+)) + ((3 × (% cells at 3))]

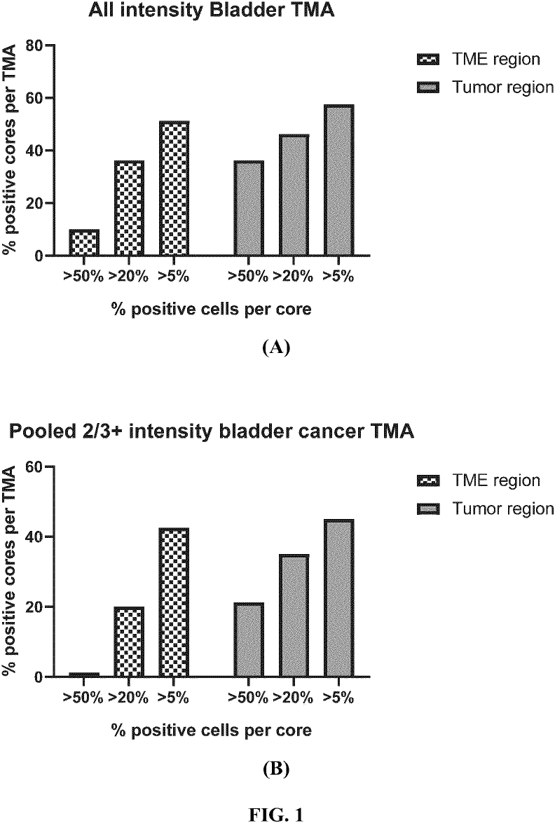

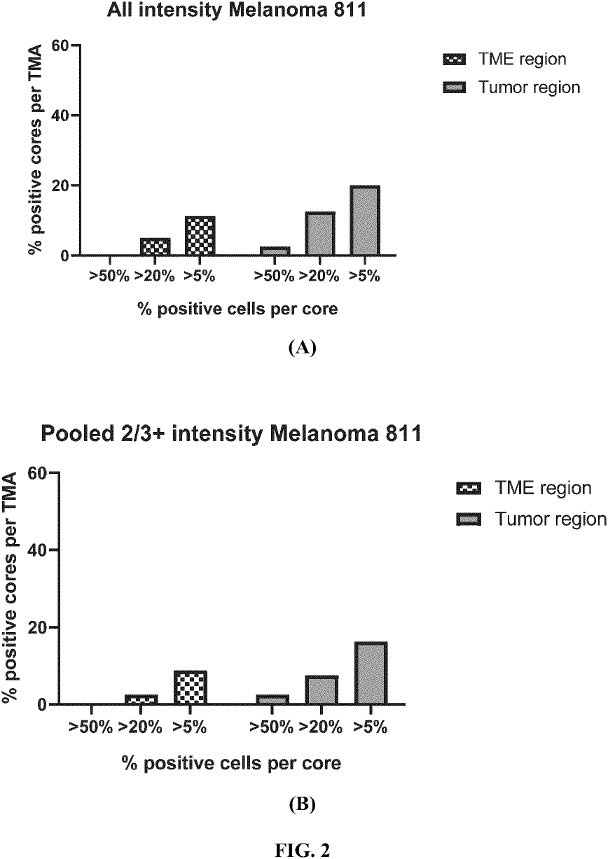

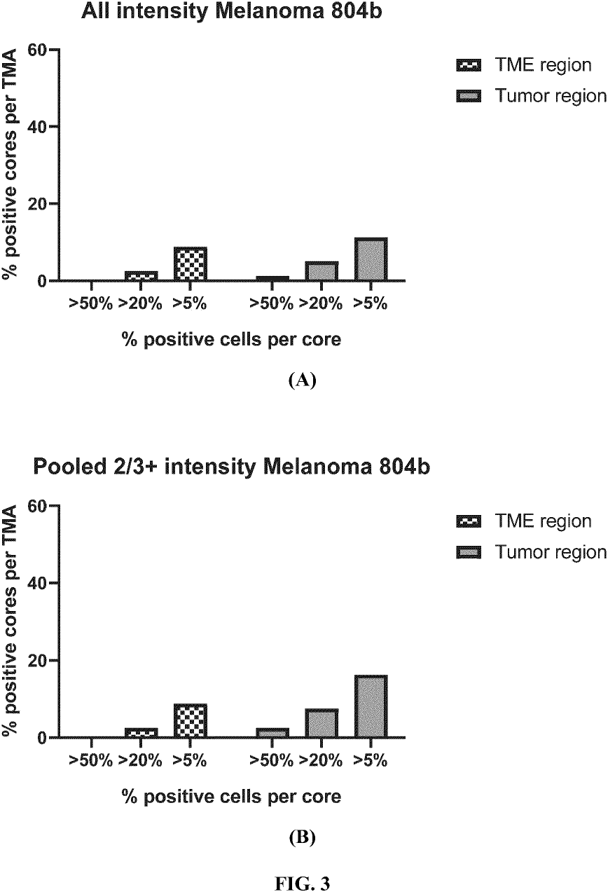

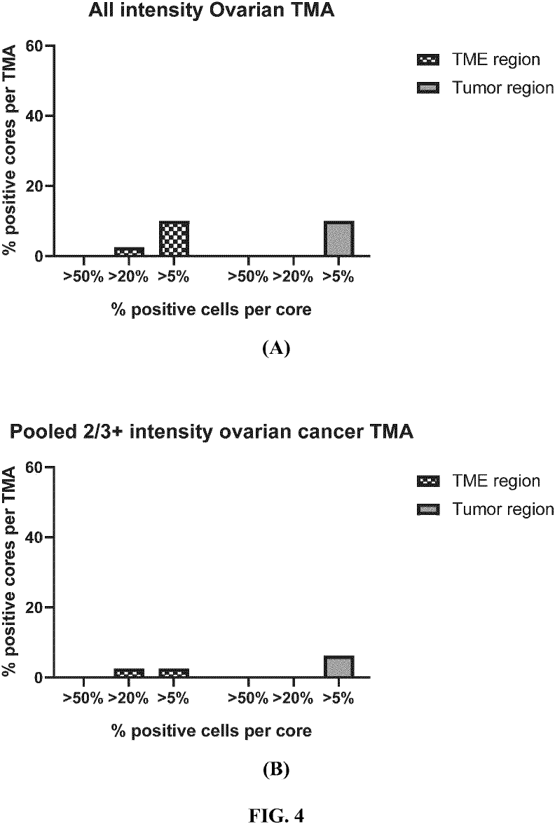

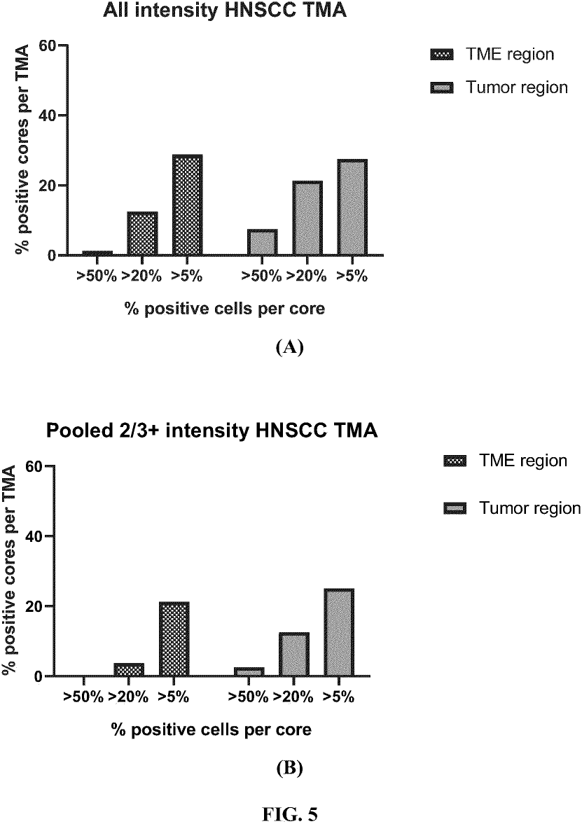

CTA scoring of AHR nuclear staining in bladder cancer, melanoma, ovarian cancer, and HNSCC patients are shown in Tables 1-10 below and in FIGS. 1-5. H-scores of bladder cancer, melanoma, ovarian cancer, and HNSCC are shown in FIG. 6.

Table 1

Bladder cancer CTA scoring of AHR nuclear staining
Bladder TMA, all-intensity

| % cells positive per core | # of TME+ cores | # of Tumor+ cores | # of total cores in the TMA | % TME+ cores | % Tumor+ cores |
|---|---|---|---|---|---|
| >50% | 8 | 29 | 80 | 10 | 36.25 |
| >20% | 29 | 37 | 80 | 36.25 | 46.25 |
| >5% | 41 | 46 | 80 | 51.25 | 57.5 |

Table 2

Melanoma TMA (811) CTA scoring of AHR nuclear staining
Melanoma TMA (811), all-intensity

| % cells positive per core | # of TME+ cores | # of Tumor+ cores | # of total cores in the TMA | % TME+ cores | % Tumor+ cores |
|---|---|---|---|---|---|
| >50% | 0 | 2 | 80 | 0 | 2.5 |
| >20% | 4 | 10 | 80 | 5 | 12.5 |
| >5% | 9 | 16 | 80 | 11.25 | 20 |

Table 3

Melanoma TMA (804b) CTA scoring of AHR nuclear staining
Melanoma TMA (804b), all-intensity

| % cells positive per core | # of TME+ cores | # of Tumor+ cores | # of total cores in the TMA | % TME+ cores | % Tumor+ cores |
|---|---|---|---|---|---|
| >50% | 0 | 1 | 80 | 0 | 1.25 |
| >20% | 2 | 4 | 80 | 2.5 | 5 |
| >5% | 7 | 9 | 80 | 8.75 | 11.25 |

Table 4

Ovarian cancer CTA scoring of AHR nuclear staining
Ovarian TMA, all-intensity

| % cells positive per core | # of TME+ cores | # of Tumor+ cores | # of total cores in the TMA | % TME+ cores | % Tumor+ cores |
|---|---|---|---|---|---|
| >50% | 0 | 0 | 80 | 0 | 0 |
| >20% | 2 | 0 | 80 | 2.5 | 0 |
| >5% | 8 | 8 | 80 | 10 | 10 |

Table 5

HNSCC CTA scoring of AHR nuclear staining
HNSCC TMA, all-intensity

| % cells positive per core | # of TME+ cores | # of Tumor+ cores | # of total cores in the TMA | % TME+ cores | % Tumor+ cores |
|---|---|---|---|---|---|
| >50% | 1 | 6 | 80 | 1.25 | 7.5 |
| >20% | 10 | 17 | 80 | 12.5 | 21.25 |
| >5% | 23 | 22 | 80 | 28.75 | 27.5 |

Table 6

Bladder cancer CTA scoring of AHR nuclear staining
Bladder TMA, pooled 2+ 3+ intensity

| % cells positive per core | # of TME+ cores | # of Tumor+ cores | # of total cores in the TMA | % TME+ cores | % Tumor+ cores |
|---|---|---|---|---|---|
| >50% | 1 | 17 | 80 | 1.25 | 21.25 |
| >20% | 16 | 28 | 80 | 20 | 35 |
| >5% | 34 | 36 | 80 | 42.5 | 45 |

Table 7

Melanoma TMA (811) CTA scoring of AHR nuclear staining
Melanoma TMA (811), pooled 2+ 3+ intensity

| % cells positive per core | # of TME+ cores | # of Tumor+ cores | # of total cores in the TMA | % TME+ cores | %Tumor+ cores |
|---|---|---|---|---|---|
| >50% | 0 | 2 | 80 | 0 | 2.5 |
| >20% | 2 | 6 | 80 | 2.5 | 7.5 |
| >5% | 7 | 13 | 80 | 8.75 | 16.25 |

Table 8

Melanoma TMA (804b) CTA scoring of AHR nuclear staining
Melanoma TMA (804b), pooled 2+ 3+ intensity

| % cells positive per core | # of TME+ cores | # of Tumor+ cores | # of total cores in the TMA | % TME+ cores | % Tumor+ cores |
|---|---|---|---|---|---|
| >50% | 0 | 0 | 80 | 0 | 0 |
| >20% | 0 | 2 | 80 | 0 | 2.5 |
| >5% | 4 | 5 | 80 | 5 | 6.25 |

Table 9

Ovarian cancer CTA scoring of AHR nuclear staining
Ovarian TMA, pooled 2+ 3+ intensity

| % cells positive per core | # of TME+ cores | # of Tumor+ cores | # of total cores in the TMA | % TME+ cores | % Tumor+ cores |
|---|---|---|---|---|---|
| >50% | 0 | 0 | 80 | 0 | 0 |
| >20% | 2 | 0 | 80 | 2.5 | 0 |
| >5% | 2 | 5 | 80 | 2.5 | 6.25 |

Table 10

HNSCC CTA scoring of AHR nuclear staining
HNSCC TMA, pooled 2+ 3+ intensity

| % cells positive per core | # of TME+ cores | # of Tumor+ cores | # of total cores in the TMA | % TME+ cores | % Tumor+ cores |
|---|---|---|---|---|---|
| >50% | 0 | 2 | 80 | 0 | 2.5 |
| >20% | 3 | 10 | 80 | 3.75 | 12.5 |
| >5% | 17 | 20 | 80 | 21.25 | 25 |

While we have described a number of embodiments of this invention, it is apparent that our basic examples may be altered to provide other embodiments that utilize the compounds and methods of this invention. Therefore, it will be appreciated that the scope of this invention is to be defined by the application and claims rather than by the specific embodiments that have been represented by way of example.

1. A method of treating cancer, comprising selecting a patient who is AHR nuclear positive, and administering to the patient a therapeutically effective amount of an AHR inhibitor.

2. The method of claim 1, wherein the cancer is selected from bladder cancer, melanoma, ovarian cancer, and HNSCC.

3. The method of claim 1 or 2, wherein selecting a patient who is AHR nuclear positive comprises IHC staining a tumor biopsy core of a patient.

4. The method of claim 3, wherein selecting a patient who is AHR nuclear positive comprises selecting a patient having about 5% or more cells in a tumor biopsy core which are AHR nuclear staining positive.

5. The method of claim 4, wherein selecting a patient who is AHR nuclear positive comprises selecting a patient having about 20% or more cells in a tumor biopsy core which are AHR nuclear staining positive.

6. The method of claim 5, wherein selecting a patient who is AHR nuclear positive comprises selecting a patient having about 50% or more cells in a tumor biopsy core which are AHR nuclear staining positive.

7. The method of any one of claims 4-6, wherein the tumor biopsy core is a tumor region of the tumor biopsy core.

8. The method of any one of claims 4-6, wherein the tumor biopsy core is a tumor microenvironment (or stroma) region of the tumor biopsy core.

9. The method of any one of claims 4-6, wherein staining positive refers to all staining intensity (including 1+, 2+, and 3+ intensities) by CTA scoring.

10. The method of any one of claims 4-6, wherein staining positive refers to pooled 2+ and 3+ staining intensity by CTA scoring.

11. The method of claim 1 or 2, wherein the AHR inhibitor is Compound A: 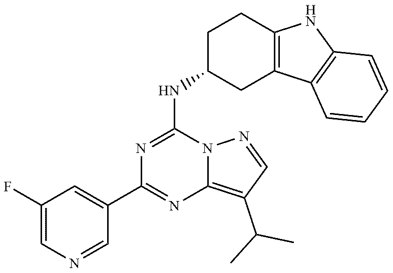 [SMILES: CC(C)c1cnn2c(N[C@@H]3CCc4[nH]c5ccccc5c4C3)nc(-c3cncc(F)c3)nc12]
or a pharmaceutically acceptable salt thereof.

12. A method of treating bladder cancer in a patient, comprising:
IHC staining a tumor tissue of a patient;
selecting a patient having about 5%, 20%, or 50%, or more cells in a tumor region of a tumor biopsy core which are AHR nuclear staining positive at all staining intensity (including 1+, 2+, and 3+ intensities) or at pooled 2+ and 3+ staining intensity by CTA scoring; and
administering to the patient a therapeutically effective amount of Compound A, or a pharmaceutically acceptable salt thereof.

13. A method of treating HNSCC in a patient, comprising:
IHC staining a tumor tissue of a patient;
selecting a patient having about 5%, 20%, or 50%, or more cells in a tumor region of a tumor biopsy core which are AHR nuclear staining positive at all staining intensity (including 1+, 2+, and 3+ intensities) or at pooled 2+ and 3+ staining intensity by CTA scoring; and
administering to the patient a therapeutically effective amount of Compound A, or a pharmaceutically acceptable salt thereof.

14. A method of treating ovarian cancer in a patient, comprising:
IHC staining a tumor tissue of a patient;
selecting a patient having about 5%, 20%, or 50%, or more cells in a tumor region of a tumor biopsy core which are AHR nuclear staining positive at all staining intensity (including 1+, 2+, and 3+ intensities) or at pooled 2+ and 3+ staining intensity by CTA scoring; and
administering to the patient a therapeutically effective amount of Compound A, or a pharmaceutically acceptable salt thereof.

15. A method of treating melanoma in a patient, comprising:
IHC staining a tumor tissue of a patient;
selecting a patient having about 5%, 20%, or 50%, or more cells in a tumor region of a tumor biopsy core which are AHR nuclear staining positive at all staining intensity (including 1+, 2+, and 3+ intensities) or at pooled 2+ and 3+ staining intensity by CTA scoring; and
administering to the patient a therapeutically effective amount of Compound A, or a pharmaceutically acceptable salt thereof.

16. A method for identifying or selecting a cancer patient who is AHR nuclear positive, comprising IHC staining a tumor tissue of a patient, and selecting a patient who is AHR nuclear staining positive.

17. The method of claim 16, wherein selecting a patient who is AHR nuclear staining positive comprises selecting a patient having about 5% or more cells in a tumor biopsy core which are staining positive.

18. The method of claim 16, wherein selecting a patient who is AHR nuclear staining positive comprises selecting a patient having about 20% or more cells in a tumor biopsy core which are staining positive.

19. The method of claim 16, wherein selecting a patient who is AHR nuclear staining positive comprises selecting a patient having about 50% or more cells in a tumor biopsy core which are staining positive.

20. The method of any one of claims 17-19, wherein the tumor biopsy core is a tumor region of the tumor biopsy core.

21. The method of any one of claims 17-19, wherein the tumor biopsy core is a tumor microenvironment (or stroma) region of the tumor biopsy core.

22. The method of any one of claims 17-19, wherein staining positive refers to all staining intensity (including 1+, 2+, and 3+ intensities) by CTA scoring.

23. The method of any one of claims 17-19, wherein staining positive refers to pooled 2+ and 3+ staining intensity by CTA scoring.

24. The method of claim 16, wherein the tumor tissue is a bladder tumor tissue, a melanoma tissue, an ovarian tumor tissue, or an HNSCC tumor tissue.

25. A method for IHC staining a tumor tissue of a patient, comprising staining a tumor tissue section using AHR monoclonal antibody FF3399.

26. The method of claim 25, further comprising measuring staining intensity in a tumor biopsy core.

27. A method of treating cancer comprising selecting a patient who has an AHR gene amplification and administering to the patient a therapeutically effective amount of an AHR antagonist.

28. The method of claim 27, wherein the selecting a patient who has an AHR gene amplification comprises using Next Generation Sequencing (NGS), RNAscope, or fluorescent in situ hybridization (FISH) to identify the AHR gene amplification.

29. The method of claims 27 or 28, wherein the AHR gene amplification is determined by assaying cells from a tumor sample.

30. The method of any one of claims 27-29, wherein about 10% of the cells from the tumor sample have at least about three copies of AHR.

31. The method of any one of claims 27-30, wherein the cancer is selected from bladder cancer, melanoma, ovarian cancer, and HNSCC.

32. The method of any one of claims 27-31, wherein the AHR antagonist is: 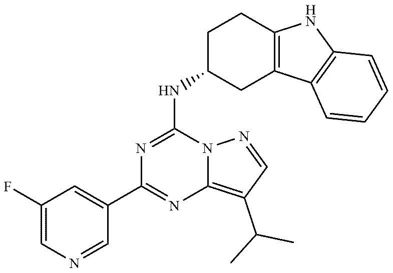 [SMILES: CC(C)c1cnn2c(N[C@@H]3CCc4[nH]c5ccccc5c4C3)nc(-c3cncc(F)c3)nc12]
or a pharmaceutically acceptable salt thereof; or
(ii) a metabolite of Compound A, or a pharmaceutically acceptable salt thereof, or a prodrug thereof.

33. A method of treating cancer in a patient who has an AHR gene amplification comprising administering to the patient a therapeutically effective amount of an AHR antagonist.

34. The method of claim 33, wherein about 10% of the cells from a tumor sample from the patient have at least about three copies of AHR.

35. The method of claims 33 or 34, wherein the cancer is selected from bladder cancer, melanoma, ovarian cancer, and HNSCC.

36. The method of any one of claims 33-35, wherein the AHR antagonist is: 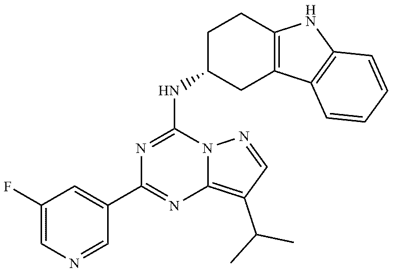 [SMILES: CC(C)c1cnn2c(N[C@@H]3CCc4[nH]c5ccccc5c4C3)nc(-c3cncc(F)c3)nc12]
or a pharmaceutically acceptable salt thereof; or
(ii) a metabolite of Compound A, or a pharmaceutically acceptable salt thereof, or a prodrug thereof.

37. A method of treating cancer in a patient who is AHR nuclear positive comprising administering to the patient a therapeutically effective amount of an AHR antagonist.

38. The method of claim 37, wherein the patient who is AHR nuclear positive has about 5% or more cells in a tumor sample which are AHR nuclear staining positive.

39. The method of claim 38, wherein the tumor sample is a tumor biopsy core of the patient.

40. The method of any one of claims 37-39, wherein the cancer is selected from bladder cancer, melanoma, ovarian cancer, and HNSCC.

41. The method of any one of claims 37-40, wherein the AHR antagonist is: 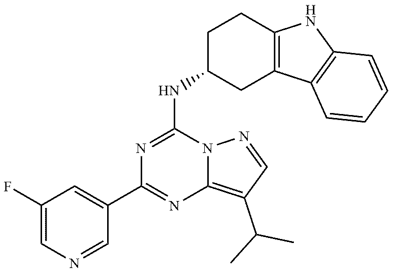 [SMILES: CC(C)c1cnn2c(N[C@@H]3CCc4[nH]c5ccccc5c4C3)nc(-c3cncc(F)c3)nc12]
or a pharmaceutically acceptable salt thereof; or
(ii) a metabolite of Compound A, or a pharmaceutically acceptable salt thereof, or a prodrug thereof.
